(19) United States
(12) Patent Application Publication
(10) Pub. No.: US 2017/0035049 A1
(43) Pub. Date: Feb. 9, 2017

(54) BIOCIDAL PRODUCTS

(71) Applicant: SALVECO, Neuvillers Sur Fave (FR)

(72) Inventor: Stephan AUBERGER, Neuvillers Sur Fave (FR)

(21) Appl. No.: 15/303,099

(22) PCT Filed: Apr. 9, 2015
(86) PCT No.: PCT/FR2015/050954

§ 371 Date: Oct. 10, 2016

(30) Foreign Application Priority Data
Apr. 10, 2014 (FR) 1453179

(51) Int. Cl. A01N 37/36; A01N 25/30; A01N 25/08

(52) U.S. Cl. CPC A01N 37/36; A01N 25/08; A01N 25/30

(57) ABSTRACT

The biocidal products do not pose any particular risks to the environment or health. Indeed, the active substances and additives that constitute the biocidal products according to the invention are of plant, agricultural, and renewable origin, and are furthermore completely biodegradable. The concentrated biodegradable biocidal formulation includes compounds from renewable plant resources, such as at least lactic acid and/or citric acid in a proportion of 76 wt % to 98 wt % relative to the total weight of the formulation; and at least one sodium and/or potassium carboxylate including 5 to 16 carbon atoms serving as an anionic surfactant in a proportion of 2 wt % to 24 wt % relative to the total weight of the formulation.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

1. Field of the Invention

The present invention relates to a new generation of biocidal products that poses no chronic risks to and very few acute impacts on health and the environment.

2. Description of Related Art including Information Disclosed Under 37 CFR 1.97 and 37 CFR 1.98

Indeed, the active substances and the additives the biocidal formulations according to the invention are comprised of are of plant origin, preferably of agricultural origin, and renewable, and are furthermore completely, naturally and quickly biodegradable.

The various applications of these new biocidal products are also claimed.

Biocides are commonly used in a wide variety of products including household and/or industrial disinfectants, insecticides, wood treatment products, etc.

These products can be used to clean and disinfect all kinds of surfaces, including the skin.

Biocidal products are aimed at destroying, repelling or making ineffective the undesirable organisms, whether or not deemed harmful, such as namely bacteria, viruses, fungi, at preventing their action or at fighting them in any other way by means of a chemical or biological action.

These categories of products are chemically or biologically active products and are therefore likely to have harmful effects on humans, animals and/or the environment.

More in particular, a biocide is generally characterized by its field of application: bactericidal, fungicidal, antiviral and/or insecticide.

Bactericides have an action against determined Gram+ and/or Gram− bacteria. Fungicides have an action against determined fungi and determined yeasts. As regards the antiviral biocides, they have an action against determined viruses.

Therefore, the active biocides can present a major risk to human and animal health, but also to the environment; indeed, from an etymological point of view “bios” means “life” and “-cide” means “which kills.” Indeed, the active biocides can act for a long time, spread, be partially degraded, or not be degraded at all depending on their nature, and they are then considered (bio)accumulative and toxic.

The active substances the biocides are comprised of are generally active ingredients of petrochemical origin.

A few rare substances of plant origin can be listed, such as ethanol or essential oils having bacteriostatic and limited properties. In addition, these substances are out of the professional markets.

Finally, biocides can also include a mixture of the last two categories of components, i.e. active ingredients of petrochemical origin and substances of plant origin, in order to achieve the desired efficiency.

Thus, in the state of the art are known the derivatives of quaternary ammonium, which represent a major class of the biocide substances of chemical origin, which are used in surface disinfectants and for hand hygiene.

Other substances are also used, such as sodium hypochlorite (bleach), di- or tri-amines or 5-chloro-2-(2,4 dichlorphenoxy)phenol (Triclosan®).

Their regulatory classification as regards their critical, corrosive, sensitizing effects and those very toxic on aquatic organisms, as well as the suspicion of a CMR (Carcinogen, Mutagen, Reprotoxic) effect and of endocrine disruptor of some representatives of this class, requires greatest caution for the use of these products.

An international awareness in this respect has led to the adoption of European Regulations (REACH: 1907/2006 and Biocides: 528/2012), which aim at restricting the marketing of hazardous chemicals and at strictly regulating their use.

Indeed, the market of biocidal products for Europe represents a volume of 300,000 to 750,000 tons per year (Study of the impact of the Biocides 98/8/EC-2007 Directive), which corresponds to an annual global market of 7 billion euros.

There exists therefore a considerable interest in developing new biocides to fight against undesired microorganisms. These biocides should have the capacity of being fully degraded naturally after their use, so as to reduce the risks to humans, animals and the environment, while limiting the induced resistances.

In particular, to humans and animals, these risks can be “direct”, related to the so-called acute toxicity, and especially “indirect”, related to the chronic toxicity.

In addition, the biocidal products must be effective at low concentration so as to provide industrial and economic solutions in line with the economic positions of the sanitary markets.

In order to provide biocidal products that are not toxic and/or harmful to humans and the environment, and that are also biodegradable and biocompatible for humans, biocides of natural origin have then been developed.

Indeed, biocidal products of plant origin seem most appropriate to meet these combined objectives.

The majority of the biocidal solutions of plant origin contain a washing base combined with a disinfectant base. However, these products have not the same biocidal effectiveness or the same ease of use (dose/time/action-spectrum ratio) as the biocidal solutions containing active and/or chemical and synthetic additives.

There is therefore a need to develop a sustainable and safe alternative to the use of chemical and synthetic products, and which permits to achieve a biocidal effectiveness and ease of use of the product to be diluted.

From WO 2012/114039 is already known a biocide composition comprising:
between 0.01% and 20% chelating agent;
between 0.03% and 25% non-ionic surfactants such as polyols, and preferably between 0.03% and 25% non-ionic surfactants such as glycosides polyglycerol esters or sorbitan esters;
between 0.03% and 25% anionic surfactants, preferably such as carboxylates or polycarboxylates;
between 0.1% and 75% of one or more organic acids;
between 0.001% and 8% natural perfume.

This composition, although being of plant origin and having a biocidal activity on determined pathogens, can however be further improved.

### BRIEF SUMMARY OF THE INVENTION

The Indeed, the composition described in this document is not optimally effective against undesired organisms and likely to be pathogenic, for example determined strains of fungi or also determined undesirable or pathogenic viruses.

The present invention thus relates to a new generation of biocidal formulations for cleaning and disinfecting all kinds of surfaces.

Their impact on the environment is very limited, even null, and they are in no case considered as critical. This results from the toxicological data of the components and the mixture, which are significantly below the imposed critical classification thresholds.

Indeed, these biocidal formulations are derived from renewable plant resources, preferably from agricultural resources and they are fully biodegradable on the entire formulation.

The active ingredient of the biocidal formulations according to the invention consists of at least one organic acid of plant origin. This organic acid can be derived directly from a plant resource, preferably an agricultural resource, or a transformed agricultural resource.

### BRIEF DESCRIPTION OF THE SEVERAL VIEWS OF THE DRAWINGS

Other features and advantages of the invention will emerge from the following detailed description of non-limiting embodiments of the invention, in reference to the appended FIGURE. The FIGURE is a graph illustration of percentages of degration over twenty eight days.

### DETAILED DESCRIPTION OF THE INVENTION

As shown in the figures of the attached drawing, the invention relates to a lifting device 1, also called lifting column, in particular designed for lifting vehicles.

Such a device 1 comprises a column 2 forming a frame and extending substantially vertically above a base 3.

The present invention describes a biodegradable concentrated biocidal formulation including compounds derived from renewable plant resources, said formulation being characterized in that it includes at least the following compounds:
at least one organic acid in a proportion between 76 and 98 wt % based on the total weight of the formulation;
at least one anionic surfactant in a proportion between 2 and 24 wt %, based on the total weight of the formulation.

Advantageously, the proportion of organic acid is between 76 and 92%, and more advantageously between 76 and 84%. As regards the proportion of anionic surfactant, it is preferably between 3 and 15% preferably between 5 and 10″k

The biocidal formulation according to the invention includes compounds deriving from plant resources, namely agricultural resources, which are completely renewable. Accordingly, said biocidal formulation is completely biodegradable.

More specifically, the composition is 100% biodegradable according to the results of the tests relating to the OCDE experimental protocol.

In addition to a significantly reduced impact on the environment compared to the state of the art, the biocidal formulations according to the invention have an innovative effect of making the biocidal active ingredient biologically available thanks to suitable surfactant structures and as a result of physical-chemical changes of the mixture, which results into a desired combination of the components and effectiveness permitting to substitute, at equal concentration, the chemical and hazardous products on the market.

This biocidal formulation has an effective action against determined undesired or pathogenic organisms, and an improved effect compared to the biocidal formulations known in the prior art. The action of the formulation is illustrated in the examples below.

In particular, this biocidal formulation permits to fight fungi belonging to the Aspergillus Niger species, which is likely to be toxic and pathogenic, namely causing pulmonary mycoses in humans.

The formulation according to the invention has in addition a biocidal activity, namely a virucidal activity, against virus strains belonging to the family of the adenoviruses, whereby the latter can be responsible for diseases such as pharyngitis, pneumonia, or also conjunctivitis. The virucidal effect has also been demonstrated against rotaviruses, responsible namely for gastroenteritis.

As regards the organic acids usable in the composition of the biocidal formulations according to the present invention, we will namely cite citric acid, lactic acid, succinic acid, pyruvic acid, glycolic acid.

The choice of the organic acid occurs depending on the field of application of the biocidal formulations and the target organisms to be eliminated or the development of which one wants to slow down. In a particular embodiment, the organic acid or acids are lactic acid and citric acid.

In an even more preferred embodiment, only lactic acid is used.

Indeed, lactic acid is one of the indispensable elements of the human metabolism, but also of the metabolism of animals and microorganisms. Lactic acid has inherently properties of interference on the microorganisms, since it acts directly on their pH and their energy cycle. Furthermore, it also has an antimicrobial action by inhibiting their growth (for Escherichia Coli among others).

In addition to the organic acid, the concentrated biocidal formulation according to the invention also includes at least one anionic surfactant.

The latter is advantageously selected among the sodium carboxylates comprising a number of carbon atoms between 5 and 16.

The anionic surfactant may also consist of a potassium carboxylate comprising a number of carbon atoms between 5 and 16.

Preferably, the carbon chain of the anionic surfactants is derived from fatty alcohol or from herbal fatty acid.

Thus, for example, the anionic surfactant consists of an alkyl carboxylate such as sodium caprate, sodium caprylate, sodium aurate, C8-C16 sodium, ammonium or potassium alkyl sulphate, an carboxylated fatty acid ester.

The anionic surfactant may also consist of acidic species derived from these salts.

The concentrated biocide formulation according to the invention may also include other ingredients.

Thus, said formulation may include, in addition to the organic acid and the anionic surfactant, at least one non-ionic surfactant and/or at least one natural perfume and/or at least one chelating agent.

The non-ionic surfactants or other components such as essential oils or chelating agents that may enter into the composition of the biocidal product according to the invention are selected depending on the type of efficiency and application wanted: a judicious mixture is necessary in order not to disturb or change the biocidal properties initially looked for. Indeed, for example, efficient disinfectant and/or degreasing properties are necessary in the world of restoration or in the agricultural industry with little or non-toxic products in order to avoid any critical chemical contamination, namely due to the presence of chemical residues on the surfaces after the disinfection process.

In a particular embodiment, the biocidal formulation according to the invention may include at least one non-ionic surfactant, preferably in a proportion between 2 and 15% weight based on the total weight of the formulation.

Advantageously, this non-ionic surfactant is selected among the glycosides and the alkyl polyglycosides the carbon chain of which contains between 5 and 20 carbon atoms, more preferably between 5 and 18 carbon atoms and the hydrophilic group is preferably comprised of pentose or hexose residues.

Thus, preferably, the formulation according to the invention may include a non-ionic surfactant selected from octyl glucoside, decyl glucoside, lauryl glucoside, coconut glucosides, D-glucopyranose, D-xylopyranose C10-C16 alkylpolyglycosides.

The chelating agents that may enter into the composition of the biocidal formulation according to the invention are conventional agents in the field of cleaning and disinfection of surfaces for home or professional use.

They are selected among the chelating agents commonly used in the biocidal products, such as citric acid, derived from lemon juice or from the fermentation of simple or complex plant sugars, especially glucose, fructose, and sucrose, or sorbic acid, derived from rowan, or oxalic acid, derived from the roots or rhizomes of many plants (sorrel, beet, etc.), sequestering monomers or polymers such as chicory extracts, or amino acid derivatives,

In addition, the chelating agent may, in one embodiment, also be derived from the fermentation of agricultural material. This basic agricultural material may for example consist of residues of molasses permitting to obtain complexing agents such as gluconates,

Preferably, the chelating agent or agents are present at 0.1% to 10 wt % based on the total weight of the formulation.

The olfactory properties of the biocidal products according to the invention can be imparted by the presence of at least one natural perfume derived from a renewable and biodegradable natural resource.

This natural perfume can advantageously be chosen among essential oils, vegetable oils or plant extracts.

Namely and as an example, A-mint, N-mint or G-eucalyptus should be cited.

Preferably, the natural perfume or perfumes are present in the concentrated biocidal formulation at 0.01% to 10 wt % based on the total weight of the formulation.

The presence of the perfume will advantageously provide an olfactory note, without participating in the biocidal activity of the product. Thus, it can be removed in conditions where formulations without perfume are required, such as in the agro-food industry.

A formulation containing the biocidal product according to the invention may be diluted 1 to 500 times in water depending on the intended use.

The formulations according to the invention comply with the European regulations in terms of efficiency. They meet the EN1040, EN1276, EN1656, EN 1275, EN 13697, EN14476 and EN1650+A1 standards in dirty or clean conditions.

In addition, these effective formulations are without any particular danger to humans and without any harm to the environment.

The biocidal formulations according to the invention are likely to be used at dilution rates and for durations of action comparable to those required by the professional market. In addition, they meet the biocidal efficacy requirements, without any particular danger for the application, are biodegradable and free of eco-toxicity after removal.

In a preferred embodiment, the biocidal formulations according to the invention are provided in concentrated liquid form. They may be highly diluted, for example 50 to 500 times, namely in water, and very well maintain their activities depending on the application being contemplated.

Thus, the present invention also relates to an aqueous liquid composition comprising at least the biocidal formulation described above and water, and all dilutions in water of said composition to a minimum concentration of 0.2% of said composition.

The rest of the composition comprises at least water. Thus, in a particular embodiment, the aqueous liquid composition according to the invention may also include other ingredients in order to improve the cleaning and/or organoleptic properties, without however changing the biocidal activity of said formulation.

In a preferred exemplary embodiment, the biocidal formulation according to the invention is present, in said aqueous liquid composition, in a proportion between 0.2 and 2 wt % based on the total weight of said composition.

In other words, said formulation can advantageously be diluted between 50 and 500 times and still be active on undesired organisms one wants to eliminate.

More preferably, the biocidal formulation according to the invention is present, in said aqueous liquid composition, in a proportion between 1 and 2% weight, more preferably between 1.5 and 2 wt %, based on the total weight of said composition.

The following examples permit to illustrate this efficiency, especially against determined pathogenic or undesirable strains of yeasts, fungi and bacteria, but also against determined pathogenic viruses.

In particular, the biocidal formulations are particularly interesting, since they remain effective even at high dilution rates, and have an effectiveness comparable to more concentrated formulations but which are less attractive from an economic point of view.

The applications contemplated for the biocidal formulations according to the invention are based on a disinfectant, but also detergent, degreasing, emollient, olfactory action, etc. . . . .

As examples should be cited, but without this list being exhaustive, an application of the formulations according to the invention to the anti-foam products for cleaning masonry; products for cleaning the pontoons and boat hulls from mosses and algae that colonize them; disinfecting detergent solutions for all types of surfaces; solutions for hand hygiene through washing or friction; disinfecting solutions for foodstuff; solutions for disinfecting premises, buildings and transportation and farming equipment; solutions for disinfecting the surfaces for the agro-food, cosmetic or pharmaceutical industry; solutions for disinfecting premises and/or transportation of domestic waste, solutions for disinfecting milking equipment, teats, animal litter.

In a particular embodiment, the biocidal formulations according to the invention are used for hand hygiene, through washing or friction, in liquid or semi-liquid form, with or without rinsing with water.

The present invention also relates to a substrate impregnated with a concentrated biocidal formulation according to the invention, permitting the application of said formulation to a surface to be cleaned and disinfected.

This substrate may consist of a tissue, a wipe or towel.

On its substrate, the formulation according to the invention may be pure and dehydrated and be activated when in the presence of water or also already be diluted and ready for use.

In other words, the substrate can also be impregnated with the aqueous liquid composition as described above, said composition including at least the biocidal formulation and water, said formulation being present in a proportion between 0.2% and 2 wt % based on the total weight of said composition.

Example of Biocidal Formulation:

In an exemplary embodiment, the biocidal formulation according to the invention has the following compounds, in the following proportions:
2.4% non-ionic surfactants such as glycosides;
5% anionic surfactants such as fatty acid salts with a chain containing 8 to 14 carbon atoms such as sodium cocoate;
91% lactic acid
1.6% water.

This formula is only classified as irritant according to regulation in force, just like the lactic acid used in said formulation. Such a classification permits it to pass through heavy and expensive instruction files for its marketing. In addition, this formulation is without any danger for humans or the environment compared to the substances of chemical origins that are sold on the market.

This formulation is diluted depending on the surfaces to be cleaned, sanitized and the disinfecting efficacy looked for. The active diluted formula permitting a surface disinfection is without any known and/or regulated danger.

In a second exemplary embodiment, the biocidal formulation according to the invention is comprised of the following:
76% lactic acid;
24% anionic surfactants such as fatty acid salts with a chain containing 8 to 10 carbon atoms such as sodium alkyl carboxylate.

In a third exemplary embodiment, the biocidal formulation according to the invention is comprised of the following:
80% lactic acid;
5% anionic surfactants such as salts fatty acids such as sodium cocoate;
1% perfuming composition based on plant extracts;
14% water.

In a fourth exemplary embodiment, the biocide formulation according to the invention is comprised of the following:
76% lactic acid;
10% non-ionic surfactant such as alkyl polyglycosides;
10% anionic surfactant such as C10-C12 carboxylate;
4% water,

Efficacy Tests of the Biocidal Formulation

The biocidal formulation that was tested is the one specified above, which includes:
2,4% non-ionic surfactants such as C10-C16 alkyl polyglycosides;
5% anionic surfactants such as fatty acid salts such as sodium caprylate;
91% lactic acid;
1.6% water.

1. Yeasts—Candida albicans

This quantitative suspension test for evaluating the basic yeasticidal activity of the formulation according to the invention is performed by a Microbiology Laboratory. This test is performed in dirty conditions and in additional conditions.

Method: A dilution/neutralization method is applied to a formulation according to the invention. The neutralizer is comprised of sodium chloride (0.85% W/V), Na₂CO₃ (0.1%), supplemented with distilled water.

The formulation according to the invention is diluted with physiological water at the following concentrations: 1%, 1.2%, 1.5% and 2%.

The tests are performed on a Candida albicans strain (ATCC 90028) at a temperature of 20° C. (±1° C.). The contact period is limited to 5 minutes (±10 seconds),

The interfering substance is bovine albumin (fraction V) at 3.00 g/l. This constitutes the dirt condition.

The tests were incubated at a temperature of 25.0° C. (±0.2° C.) for 48 h.

Results: in accordance with the provisions of the NF EN 1275 standard (April 2006), the formula according to the invention has a yeasticidal activity in dirty conditions (3,00 g/l of bovine albumin) after 5 minutes of contact at 20° C. for a Candida albicans reference strain for the concentration of 1.2%.

2. Fungi—Aspergillus Niger

The fungicidal activity of the biocidal formulation according to the invention was also tested, more particularly on a fungus belonging to the Aspergillus Niger species.

The biocidal formulation was diluted at a concentration of 2% in water,

The tests were conducted at a temperature of 20° C. (±1° C.). Two contact periods were tested; 15 min and 30 min.

The interfering substance is bovine albumin (fraction V) at 0.30 g/l. This constitutes the clean condition.

The tests were incubated at a temperature of 30° C. for a 2 days' period.

Results: The formulation diluted at 2% has a fungicidal activity against the Aspergillus Niger strain after a period of contact of 30 min at a temperature of 20° C. and in clean conditions (0.30 g/l of bovine albumin),

The results obtained for C. albicans and A. niger are listed in the table below.

TABLE 1
Tests of fungicidal activity according to EN1650, dirty (3 g/L BSA) and
clean conditions (0.3 g/L BSA), 20° C.

| | | test | Period | | Fungi- | |
|---|---|---|---|---|---|---|
| | | concentration | of | Reduction | cide | BSA |
| Strain | Tested lot | (w/w) | contact | log | activity | (g/L) |
| Candida | 0182/ | 2% | 5 min | R > 4.22 | YES | 3 |
| albicans | 2013 | 1.5% | 5 min | R > 4.22 | YES | 3 |
| | | 1.2% | 5 min | R > 4.07 | YES | 3 |
| | | 1% | 5 min | R < 2.70 | NO | 3 |
| Aspergillus | 0346A/ | 2% | 30 min | R = 4.125 | YES | 0.3 |
| niger | 2013 | | | | | |
| | 0395A/ | 2% | 15 min | R = 4.25 | NO | 0.3 |
| | 2013 | | | | | |

3. Virus—Rotavirus and Adenovirus

This test of virucidal efficacy is performed according to the methodology of the NF EN 14476+A1 standard (January, 2007) on two viruses.

a) Rotavirus

The first virus being tested is a Rotavirus (ATCC VR-1290 strain) and the tests were conducted on three samples of disinfecting product ready for use, according to the formulation of the present invention diluted at 2% in water.

Method: The test is performed at 20° C. (±1° C. The contact period is 15 minutes. The samples are diluted in sterile distilled water. The interfering substance is bovine albumin (BSA 3.00 g/l).

The virus strain was tested on MA-104 cells at 36.5° C. under 5% CO₂.

The technique for stopping the virucidal action is the addition of a solution containing Na₂CO₃, at 70 g/l, pH 10.85 and 10 ml of stopping solution for 10 ml of test solution. Such a solution permits to stop the action of the disinfectant on the virus and to measure the effectiveness of the biocidal formulation being tested.

The virus was titrated in PFU/ml (range forming units) or in number of infectious virus particles per ml and more commonly in logarithm of the virus titer.

Results: The three samples of biocidal formulation according to the invention have a virucidal activity on the Rotavirus strain at a concentration of 2% for 15 minutes of contact.

b) Adenovirus

The second virus being tested is an Adenovirus (Adenoid 75 ATCC VR-5 strain).

The biocidal formulation according to the invention is diluted at 2% in water and the test method is the same as the one used on the Rotavirus and described above.

However, the contact period is 60 min.

The viral strain was tested on HEp-2 cells at 36.5° C. under 5% CO₂.

Results: The three samples of biocidal formulation according to the invention have a virucid activity on the Adenovirus strain at a concentration of 2% for 60 minutes of contact.

The results obtained for the tests of virucidal activity are listed in the table below:

TABLE 2
Test of virucidal activity according to EN 14476 + A1 , clean
conditions (0.3 g/L BSA), 20° C.

| | | Test | | | | |
|---|---|---|---|---|---|---|
| | | concen- | Period | Re- | Viruci- | |
| | | tration | of | duction | dal | BSA |
| Viral strain | Tested lot | (w/w) | contact | log | activity | (g/L) |
| Rotavirus | 0212/2013 | 2% | 15 min | R = 4.25 | YES | 0.3 |
| ATCC VR- | 0226/2013 | 2% | 15 min | R = 4.375 | YES | 0.3 |
| 1290 strain | 0227/2013 | 2% | 15 min | R = 4.125 | YES | 0.3 |
| Adenovirus | 0395A/ | 2% | 60 min | R = 4.125 | YES | 0.3 |
| adenoid | 2013 | | | | | |
| 75ATCC | 0406A/ | 2% | 60 min | R = 4.25 | YES | 0.3 |
| VR-5 strain | 2013 | | | | | |
| | 04066/ | 2% | 60 min | R = 4.125 | YES | 0.3 |
| | 2013 | | | | | |

It was also shown that the present biocidal formulation is effective at a concentration of 1% on the wrapped viruses with DNA genome, such as those belonging to the Herpesviridae family. In addition, the biocidal formulation is also effective at 1% on influenza viruses.

4. Bacteria

This quantitative suspension test for evaluating the bactericidal activity of the formulations according to the present invention is performed by a Microbiology Laboratory.

Method: A dilution/neutralization method is applied to a formulation according to the invention. The neutralizer is comprised of sodium chloride (0.85% W/V), Na₂CO₃ (0.1%), supplemented with distilled water. This dilution/neutralization method permits to stop the biocidal action of the product at the end of the tested contact period.

The formulation according to the invention is diluted in physiological water at the following concentrations; 0.5%, 1.2% and 1.5%.

The tests are performed on strains of Staphylococcus aureus (ATCC 6538), Escherichia coil (ATCC 10536), Enterococcus hirae (ATCC 10541) and Pseudomonas aeruginosa (ATCC 15442) at a temperature of 20° C. (±1° C.). The contact period is limited to 5 minutes (±10 seconds).

The interfering substance is bovine albumin (fraction V) at 3.00 g/l. This constitutes the dirt condition.

The incubation is carried out at a temperature of 37.0° C. (±0.2° C.) for 24 h.

Results: According to the indications of the NF EN 1276 standard (October 1997), a concentrated disinfection formula according to the formulation of the present invention has a bactericidal activity in dirty conditions (3.00 g/l of bovine albumin) after 5 minutes of contact at 20° C. on the 4 reference strains tested for the test concentration of 1%. The limiting microorganism is Staphylococcus aureus (ATCC 6538).

The results of the bactericidal tests are shown in Table 3 below.

TABLE 3
Tests of activity according to EN 1276, dirty conditions
(3 g/L BSA), contact period 5 min, 20° C.

| | test | | test procedure at concentration | | | |
|---|---|---|---|---|---|---|
| Test | suspension | | of % (v/v) | | | |
| organisms | (N and N₀) | | | 0.5% | 1.2% | 1.5% |
| Pseudomonas | 10⁶ | 291 | Vc | >300 | 26 | <10 |
| aeruginosa | 10⁷ | 22 | | | | |
| ATCC15442 | N | 2.85E+ | Na | >3000 | 260 | <100 |
| | Ig N | 08 | Ig Na | 3.47 | 2.41 | 2.00 |
| | Ig | 8.45 | R | | 5.04 | >5.45 |
| | N₀ | 7.45 | Active | NO | YES | YES |
| Enterococcus | 10⁶ | 122 | Vc | >300 | <10 | <10 |
| hirae ATCC | 10⁷ | 12.0 | | | | |
| 10541 | N | 1.20E+ | Na | >3000 | <100 | <100 |
| | Ig N | 08 | Ig Na | 3.47 | <100 | <100 |
| | Ig | 8.08 | R | | 5.08 | >5.08 |
| | N₀ | 7.08 | Active | NO | YES | YES |
| Staphylococcus | 10⁶ | 144 | Vc | >300 | <10 | <10 |
| aureus | 10⁷ | 7.1 | | | | |
| ATCC 6538 | N | 1.44E+ | Na | >3000 | <100 | <100 |
| | Ig N | 08 | Ig Na | 3.47 | 2.00 | 2.00 |
| | Ig | 8.16 | R | | >5.16 | >5.16 |
| | N₀ | 7.16 | Active | NO | YES | YES |
| Escherichia | 10⁶ | 160 | Vc | >300 | <10 | <10 |
| coli ATCC | 10⁷ | 18 | | | | |
| 10536 | N | 1.62E+ | Na | >3000 | <100 | <100 |
| | Ig N | 08 | Ig Na | 3.47 | 2.00 | 2.00 |
| | Ig | 8.21 | R | | 5.21 | >5.21 |
| | N₀ | 7.21 | Active | NO | YES | YES |

Tests are also performed according to a standardized method (EN13697) so as to demonstrate the bactericidal and/or yeasticidal by application on surfaces.

The control surfaces being used are stainless steel disks on which is deposited a solution of bacteria and/or yeasts. They are more particularly microbial strains mentioned above. The discs were then immersed in the biocidal solution at 20° C. (+/−0.2° C.) for a contact period of 5 minutes. The residual microbial concentration is determined after incubation at 37° C. (+/−2° C.) for the bacteria or at 25° C. (+/−0.2° C.) for the yeasts.

Results: According to the indications of the EN 13697 standard (November 2001), a concentrated disinfecting formula according to the formulation of the present invention has a bactericidal and yeasticidal activity in dirty conditions (3.00 g/l of bovine albumin) after 5 minutes of contact at 20° C. for the 4 reference bacterial strains being tested and the yeast strain for the test concentration of 1%.

5. Biodegradability

This test is an evaluation in an aqueous medium of the ultimate aerobic biodegradability of a sample of the concentrated formulation according to the invention.

This test is performed according to the provisions of the OECD 301 A method using a sample containing a dissolved organic carbon content of 10 mg/l (DOC for dissolved organic carbon). The evolution of the disappearance of dissolved organic carbon is followed during a period of 28 days in the “Concentrated disinfection” test at 10 mg/l of DOC. The physical-chemical analysis is performed according to the ASTM D5291 standard.

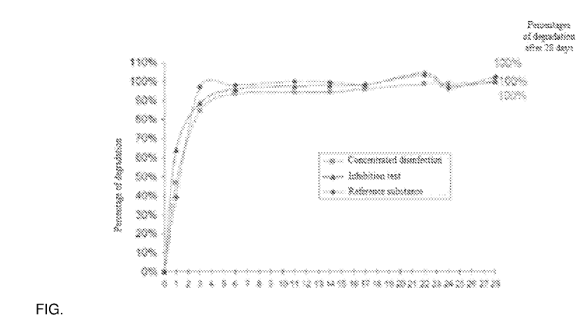

The attached FIG. 1 represents the resulting curves of degradation of the organic carbon dissolved after 28 days. The reference substance corresponds to sodium acetate. The sample referenced as “concentrated disinfection” corresponds to the biocidal formulation being tested described above and the “inhibition test” sample corresponds to a mixture between the formulation according to the invention and the sodium acetate.

Results:

The test is considered valid because:
The percentage of degradation of the reference substance (sodium acetate) is higher than 70% (99%) on the fourteenth day;
The difference between the measurement values of the DOC of the product under examination is less than 20% in each sample;
The degree of elimination of DOC in the inhibitor test, containing the test compound and the reference substance, being higher than 35% after 14 days (98%), the sample is not considered toxic for the seeding.

Conclusion:

The evaluation in an aqueous medium of the “ultimate” biodegradability of the sample tested according to the OCDE 301 A guideline gives the following results:
The maximum degree of biodegradation of the sample at 10 mg/l of DOC is equal to 100% after 28 days of incubation;
The biodegradation period (corresponding to 90% of the maximum biodegradation rate) is about 25 days,
According to the OCDE 301 A guideline, the “Concentrated disinfection” sample tested is considered as easily biodegradable (the threshold level of decrease of the DOC of 70% being acquired in an interval of 10 days after 10% of biodegradability is reached (94% at the end of the interval of 10 days on the 11^{th} day of the test),

The measured degree of biodegradability takes into consideration the entire formulation : indeed, there is no by-product from the degradation, because the formulation is exclusively comprised of carbon, oxygen and hydrogen atoms derived from plant extracts. It is a true measurement of the ultimate biodegradability.

1. A biodegradable concentrated biocidal formulation, comprising:
at least lactic acid derived from a renewable plant resource in a proportion between 76 and 98 wt %; and
at least one sodium and/or potassium carboxylate derived from a renewable plant resource comprising a number of carbon atoms between 5 and 16 acting as the anionic surfactant in a proportion between 2 and 24 wt %.

2. The concentrated biocidal formulation according to the preceding claim, wherein the lactic acid is between 76 and 92 wt % lactic acid, and wherein the carboxylate is between 3 and 15 wt % sodium and/or potassium carboxylate comprising a number of carbon atoms between 5 and 16 acting as an anionic surfactant.

3. The concentrated biocidal formulation according to claim 1, wherein the anionic surfactant consists of an alkyl carboxylate selected among sodium caprate, sodium caprylate, sodium laurate.

4. The concentrated biocidal formulation according to claim 1, further comprising a non-ionic surfactant in a proportion between 2 and 15 wt %, said non-ionic surfactant being selected among the glycosides and the alkyl polyglycosides comprising a number of carbon atoms between 5 and 20, preferably between 5 and 18 carbon atoms, the hydrophilic group being comprised of pentose or hexose.

5. The concentrated biocidal formulation according to claim 4, wherein the non-ionic surfactant is selected among the octyl glucosides, the decyl glucosides, the lauryl glucosides, the coco glucosides, D-glucopyranose, D-xylopyranose or the C10-C16 alkylpolyglycosides.

6. The concentrated biocidal formulation according to claim 1, further comprising: a natural perfume in a proportion between 0.01 and 1 wt %, said perfume being chosen among the essential oils, the plant essences and the plant extracts.

7. The concentrated biocidal formulation according to claim 1, further comprising: a chelating agent in a proportion between 0.1 and 10 wt %, said chelating agent being selected among sorbic acid derived from the sorb, oxalic acid derived from the roots or rhizomes of plants, the sequestering monomers or polymers such as the chicory extracts, and the amino acids.

8. The concentrated biocidal formulation according to claim 1, further comprising:
2.4% non-ionic surfactants such as glycosides;
5% anionic surfactants such as fatty acid salts with a chain containing 8 to 14 carbon atoms such as sodium cocoate;
91% lactic acid; and
1.6% water.

9. An aqueous liquid composition, comprising:
at least the biocidal formulation according to claim 1; and
water, wherein the biocidal formulation is present in a proportion between 0.2 and 2 wt % of the composition.

10. A substrate, comprising:
a concentrated formulation with biocidal activity according to claim 1, said substrate permitting the application of said formulation on a surface to be cleaned and disinfected.

11. The substrate impregnated with an aqueous liquid composition, according to claim 10, said substrate permitting the application of said formulation on a surface to be cleaned and disinfected.
